(19) United States
(12) Patent Application Publication
(10) Pub. No.: US 2004/0172093 A1
(43) Pub. Date: Sep. 2, 2004

(54) Apparatus for promoting nerve regeneration in paralyzed patients

(76) Inventor: Patrick D. Rummerfield, O'Fallon, IL (US)

Correspondence Address: POLSTER, LIEDER, WOODRUFF & LUCCHESI, 12412 POWERSCOURT DRIVE SUITE 200, ST. LOUIS, MO, 63131-3615, US

(21) Appl. No.: 10/355,877

(22) Filed: Jan. 31, 2003

(51) Int. Cl. A61N 1/18

(52) U.S. Cl. 607/48; 607/43

(57) ABSTRACT

In one aspect the present disclosure provides a method of rehabilitating a person who has suffered spinal cord damage including the steps of providing exercise equipment capable of exercising a person's limbs, providing functional electrical stimulation to the person's limbs to be exercised in order to operate the exercise equipment, reducing the level of functional electrical stimulation as the person's muscles tire, decreasing a resistance provided by the exercise equipment or providing assistance to maintain an acceptable speed of the exercise equipment, and completely removing functional electrical stimulation to the person's limbs and providing assistance to maintain an acceptable speed of the exercise equipment. Also provided are apparatus to carry out the method.

### RELATED APPLICATIONS

This application claims priority to U.S. patent application Ser. No. 10/252,218 filed Sep. 23, 2002 which claims priority to U.S. Provisional Patent Application No. 60/323,828 filed Sep. 21, 2001.

### TECHNICAL FIELD

The present invention relates generally to apparatus for the promotion of nerve regeneration in paralyzed patients. More specifically, it relates to an apparatus that promotes nerve regeneration by combining active and passive exercise of the patient's disabled limbs.

### BACKGROUND OF THE INVENTION

More than one-quarter of a million people currently have impaired use of their limbs due to injuries to their nervous systems. This impaired limb use, besides creating mental and physical challenges to the patient also can generate muscular atrophy, loss of bone mineral content, decubitus ulcers, urinary tract infections, muscle spasticity, impaired circulation, and reduced heart and lung capacity. Generally, impaired use is a result of a spinal injury or stroke, but can be the result of a number of conditions.

In the past, exercise for paralyzed individuals consisted of moving the patient's limbs passively in order to avoid the problems of impaired limb use. Typically, a therapist would manipulate the patient's limbs manually. However, passive exercise does induce as much blood flow or reduce muscle atrophy enough to fully avoid the problems associated with paralyzed limbs.

As result, systems were developed that utilized functional electrical stimulation to directly induce the muscles in a paralyzed limb to contract in order to perform exercise. As these systems improved, active exercise became the preferred method of exercising a paralyzed patient's limbs.

However, active exercise using functional electrical stimulation is generally used only until the muscle becomes tired. After the muscle tires, conventionally therapy is concluded, and the muscle is allowed to rest. We have unexpectedly discovered that continuing movement of the impaired limb using passive stimulus after discontinuing functional electrical stimulation promotes nerve regeneration in the affected area of the nervous system. In order to continue movement of the limb after the muscles tire, functional electrical stimulation is reduced or discontinued and passive exercise is initiated. By passively moving the affected limbs by mechanical means memories of control of the limbs are “remembered” by the nervous system. As a result, individuals with paralyzed limbs are able to gain some control of the paralyzed limb.

### SUMMARY OF THE INVENTION

In one aspect the present invention provides a method of rehabilitating a person who has suffered spinal cord damage comprising the steps of providing exercise equipment capable of exercising a person's limbs, exercised in order to operate the exercise equipment, reducing the level of functional electrical stimulation as the person's muscles tire, decreasing a resistance provided by the exercise equipment or providing assistance to maintain an acceptable speed of the exercise equipment, and completely removing functional electrical stimulation to the person's limbs and providing assistance to maintain an acceptable speed of the exercise equipment.

Another aspect of the present invention provides a bicycle for exercising limbs of a paralyzed person comprising a pair of pedals operable by the paralyzed person, a functional electrical stimulation controller, and an electric motor for providing resistance or assistance to the pedals in order to maintain a desired pedal speed. The functional electrical stimulator comprises functional electrical stimulation to muscles of the limbs of the paralyzed person in order to operate the pedals wherein the functional electrical stimulation controller is adapted for reducing the level of functional electrical stimulation as the muscles tire.

### BRIEF DESCRIPTION OF THE DRAWINGS

FIG. 1 is a side view of a bicycle for a paralyzed patient according to an embodiment of the present invention;

FIG. 2 is a perspective view of a bicycle for a paralyzed patient according to a second embodiment of the present invention; and

FIG. 3 is a side view of a gate trainer for a paralyzed patient according to an embodiment of the present invention.

### DETAILED DESCRIPTION OF THE PREFERRED EMBODIMENT

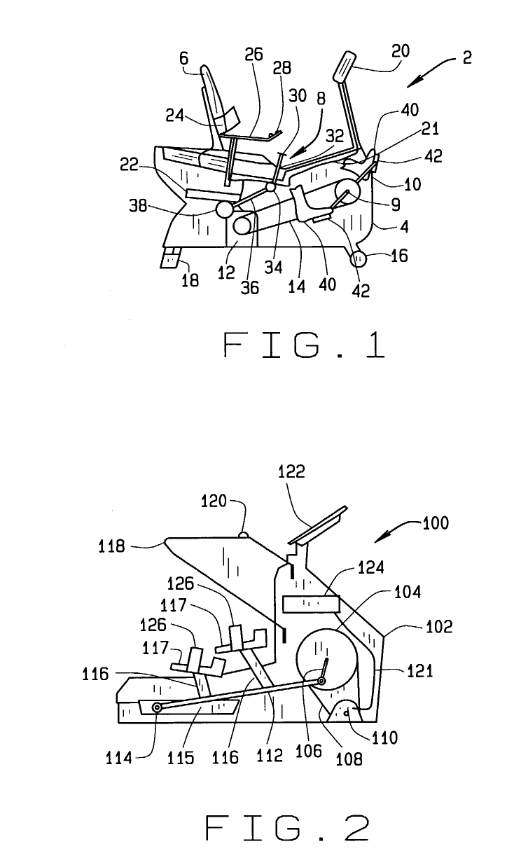

Referring to FIG. 1, there is provided an active/passive stimulation exercise trainer 2. The trainer 2 comprises a frame portion 4 to which is attached a seat 6, a pair of adjustable leg braces 8, a pulley or sprocket 9, crank arms 10 connected to the sprocket 9 and an electric motor 12 also connected to the sprocket 9 by a belt or chain 14. The frame portion 4 is maintained in an upright position by outwardly extending front and rear feet 16, 18. Also attached to the frame 4 is a bike control and readout (BCR) computer 20 attached to a functional electrical stimulation (FES) computer 22, the electric motor 12, and a control pad 28 by a cable 21. The FES computer 22 is a known device for electrical stimulation of muscles to induce organized contractions in order to move a patient's limbs. FES computers 22 are known and available from a variety of sources. The BCR computer 20 tracks stimulation current and cycle RPM and displays motor resistance, stimulation current, miles, total revolutions and RPM.

Attached to the seat 6 are a trunk support 24 and a seat belt (not shown) for providing additional support for a paralyzed patient. Also attached to the seat 6 and frame portion 4 is an armrest 26 and the control pad 28. The control pad 28 has an emergency stop button, a speed control dial or button and a start button. It is also contemplated that mounted to the control pad 28 or the BCR 20 is a microphone for receiving speech commands from the patient or therapist to be processed by the BCR computer 20 for controlling the trainer 2.

Each of the leg braces 8 has a leg support portion 30 attached to a first rod 32 that is extendable from a first clamp 34. Also attached to the first clamp 34 is a second rod 36 that is extendable from a second clamp 38. The second clamp is attached to the frame portion 4. By loosening the clamps 34, 38 and sliding the rods 32, 36 within the clamps 34, 38 and retightening the clamps 34, 38, the leg brace 8 can be adjusted to support patients of different sizes. Additionally it is contemplated that the seat 6 back can also be adjusted to support patients of different sizes. Finally boots 40 are attached to pedals 42 positioned on the ends of the crank arms 10 to fasten a patient's foot to the crank arms 10.

In operation, a paralyzed patient is seated on the chair 6 and is held by a seat belt. The patient's feet are attached to the boots 40 and the FES computer 22 is attached to the patient to begin electrical stimulation of the patient's muscles. Once FES begins, the patient's muscles begin to rotate the crank arms 10. The rotating crank 10, in turn, rotates the electric motor 12 through the chain 14. Initially, the motor 12 operates in a brake mode in order to provide resistance to the patient's muscles. The BCR computer 20 monitors the motor 12 rotation speed and controls the brake force of the motor 12 to maintain a desired RPM. As the patient's muscles begin to tire, less brake force will be required to maintain the desired RPM until, at some point, the motor 12 will switch from a brake mode to a motor mode whereby the motor 12 is providing the power required to either assist the tired muscles in rotating the crank 10 (i.e. with full or reduced FES) or to continue rotating the crank 10 in the absence of assistance from the patient's muscles (i.e. in the absence of FES). We have found that utilizing passive exercise after FES is discontinued causes nerve regeneration in the patient.

Referring to FIG. 2, there is shown and provided a second embodiment 200 of the stationary bicycle of FIG. 1. This embodiment allows a person confined to a wheelchair to use the stationary bicycle 200 without being lifted from their wheelchair to a bicycle seat, thus allowing the person to administer therapy upon the bicycle without assistance from others, in the case of a paraplegic, or with greater ease and the assistance of fewer people in the case of a quadriplegic. The stationary bicycle 200 generally comprises a platform 202 having a wheel block 204. While not shown, the wheel block 204 may also be placed in front of the wheels of the wheel chair. The wheel block 204 is adjusted to the proper location to accommodate the leg length of the user to assist the wheel locks of the user to hold the wheelchair stationary while the user pedals the bicycle 200.

The bicycle 200 further comprises pedals 206, a chain or belt 208, and a generator/motor 210 as in the first embodiment. As in the first embodiment, the user's muscles are stimulated through FES and as the muscles tire, rotation of the pedals is tapered or switched from active to passive exercise in order to move the individual's limbs. Further, there is shown a screen 212 through which a user or the user's assistant may communicate with a computer (not shown) for controlling the bicycle. The computer allows the logging of data, such as quantity of muscle action, allows the use of a digital camera to communicate with a therapist at a remote location through a telecommunication link (such as the Internet), indicate upon the screen how the patient is progressing within the therapy or progress between therapies, transmit progress data regarding the patient's progress and activity to a therapist at a remote location, allow the user to contact an Internet portal for others in a similar condition for communication, education and support. Furthermore, it is further contemplated that a virtual image of the user could be displayed on the screen showing the user pedaling the bicycle in a virtual location. For example, the user could be shown bicycling down a mountain pass in the Tour de France or taking a leisurely ride on a nature path. It is contemplated that such virtual images could provide motivation and entertainment for users while their limbs are being exercised. It is further contemplated that the control computer may implement voice recognition software to accept commands to allow the user to control the bicycle 200 without the need to physically touch buttons. Finally, it is contemplated that the computer would implement a personal computer operating system to allow the user to perform the function of a personal computer, such as surfing the Internet, writing email, drafting letters, playing games and tracking personal finances while the user's limbs are being exercised.

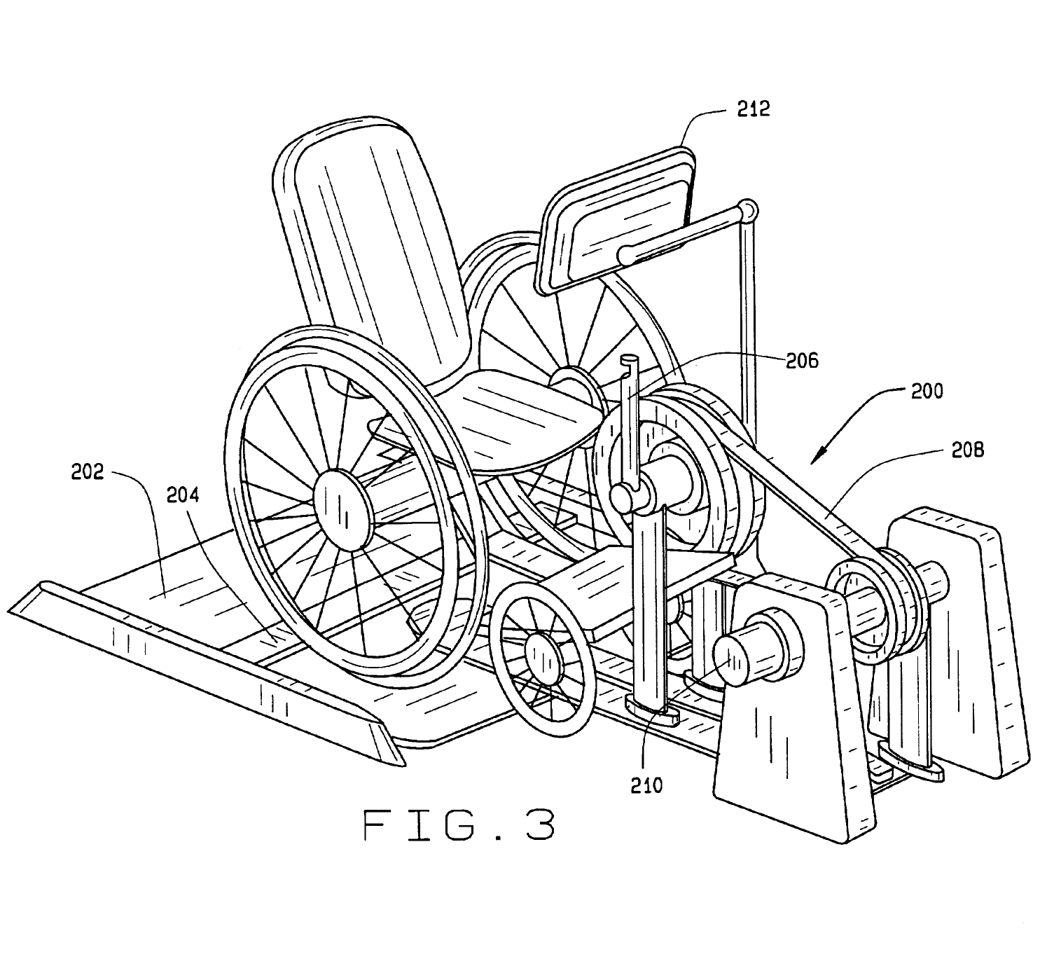

Referring to FIG. 3, there is shown a gate trainer exercise machine 100. The gate trainer exercise machine 100 simulates the act of walking for a patient. To that end, there is provided a gate trainer frame 102 on which a flywheel 104 is mounted. Opposed crank arms 106 are attached to the flywheel 104. Connected to the flywheel 104 by a belt or chain 108 is an electric motor 110. First ends of two translational motion shafts 112 are connected to the crank arms 106 on either side of the flywheel 104. Rollers 114 are attached to the opposite ends of the translational motion shafts 112.

The gate trainer frame 102 defines two slots 115 on opposite sides of the frame 102 in which the rollers 114 are trapped. The rollers 114 move along the slots 115 in translational motion with the slots 115. Also attached to each translational motion shaft 112 is a foot support rod 116 and a foot support 117. A handrail 118 is attached to the gate trainer frame 102 and has a control pad 120 with an emergency stop button, a speed control dial or button and a start button. Also attached to the frame 102 is a gate trainer control and readout (GTCR) computer 122 that is attached to a FES computer 124, the electric motor 110, and the control pad 120 by a cable 121. The GTCR computer 122 tracks stimulation current and crank or motor RPM and displays motor resistance, stimulation current, miles, total revolutions and RPM.

It is also contemplated that each foot support 117 can be height-adjustable. This can be accomplished in numerous ways, for example by providing two telescoping rods having holes drilled therethrough in place of each foot support rod 116. To maintain the two telescoping rods in the proper relationship a pin can be inserted through the holes of the rods.

The gate trainer exercise machine 100 is operated by lifting a paralyzed patient in an overhead hoist (not shown) that supports the patient's torso and allows the patient's legs to be moved freely. The patient is placed over the foot supports 116 and the patient's feet are strapped to the foot supports 116 using foot straps 126. Once the patient is in position, the motor 110 begins rotating thereby turning the flywheel 104. The flywheel 104, in turn, rotates the crank arms 106. The rotational motion of the crank 106 is then converted to translational motion by the translational motion shafts 112 and the rolling of the rollers 114 within the slots 115. However, there is also an up-and-down motion to the translation motion shafts 112 as the translational motion shafts 112 rotate with the crank 106 using the rollers 114 as a center point. The elliptical motion created by the translational motion shafts 112 is thus similar to the lifting and then stepping of a person's feet while walking.

When the patient first begins using the gate trainer exercise machine 100, FES is used to stimulate the patient's muscles in order to cause the patient to simulate walking. The motor 110 is run in a brake mode to provide resistance to the elliptical walking motion of the patient's feet on the foot supports 117. As the patient's muscles begin to tire, the RPM of the motor 110 begins to slow and less brake force is applied by the motor in order to maintain the exercise. Eventually, as the patient's muscles reach a point beyond which they are too tired to continue, the motor 110 switches from brake mode to motor mode. Thus the treatment switches from an active exercise to a passive exercise. It has been found that, like above, passive exercise of the patient's limbs awakens neural “memories” of walking in the patient's nervous system, thus rehabilitating the patient's nervous system.

While machines have been shown and described that serve the purpose of rehabilitating a person's nervous system for controlling the person's legs, the principles of the present invention apply equally to rehabilitating the nervous system for controlling a person's arm or other muscles. For example the embodiment of FIG. 1 could be easily altered to allow rotation of crank 10 by a person's arms and hands. The embodiment of FIG. 1 could be easily altered to allow elliptical rotation of a person's arms and hands. It is to be understood that the present disclosure is to be considered only as an example of the principles of the invention. This disclosure is not intended to limit the broad aspect of the invention to the illustrated embodiment.

I claim:

1. A method of rehabilitating a person who has suffered spinal cord damage comprising the steps of:
providing exercise equipment capable of exercising a person's limbs;
providing functional electrical stimulation to the person's limbs to be exercised in order to operate the exercise equipment;
reducing the level of functional electrical stimulation as the person's muscles tire;
decreasing a resistance provided by the exercise equipment or providing assistance to maintain an acceptable speed of the exercise equipment;
completely removing functional electrical stimulation to the person's limbs and providing assistance to maintain an acceptable speed of the exercise equipment.

2. The method of claim 1 wherein the step of reducing the level of functional electrical stimulation as the person's muscles tire comprises the step of continuously reducing the level of functional electrical stimulation.

3. The method of claim 1 wherein the step of reducing the level of functional electrical stimulation as the person's muscles tire comprises the step of reducing the level of functional electrical stimulation stepwise.

4. The method of claim 1 wherein the step of reducing the level of functional electrical stimulation as the person's muscles tire comprises the step of reducing the level of functional electrical stimulation to zero.

5. The method of claim 1 wherein the step of providing exercise equipment capable of exercising a person's limbs comprises the step of providing a gate trainer.

6. The method of claim 1 wherein the step of providing exercise equipment capable of exercising a person's limbs comprises the step of providing a stationary bicycle.

7. The method of claim 1 wherein the step of providing exercise equipment capable of exercising a person's limbs comprises the step of providing a stationary bicycle that can be pedaled by the user without being removed from a wheelchair.

8. A bicycle for exercising limbs of a paralyzed person comprising:
a pair of pedals operable by the paralyzed person;
a functional electrical stimulation controller comprising functional electrical stimulation to muscles of the limbs of the paralyzed person in order to operate the pedals wherein the functional electrical stimulation controller is adapted for reducing the level of functional electrical stimulation as the muscles tire; and
an electric motor for providing resistance or assistance to the pedals in order to maintain a desired pedal speed.

9. The bicycle of claim 8 wherein the functional electrical stimulation controller adapted for reducing the level of functional electrical stimulation as the muscles tire is further adapted to provide a continuous decrease in functional electrical stimulation until the muscles are no longer being stimulated.

10. The bicycle of claim 8 wherein the functional electrical stimulation controller adapted for reducing the level of functional electrical stimulation as the muscles tire is further adapted to provide a stepwise decrease in functional electrical stimulation until the muscles are no longer being stimulated.

11. The bicycle of claim 8 wherein the functional electrical stimulation controller adapted for reducing the level of functional electrical stimulation as the muscles tire is further adapted to provide an immediate discontinuance of functional electrical stimulation.

12. The bicycle of claim 8 comprising a seat into which the person is placed in order to operate the pedals.

13. The bicycle of claim 8 comprising a platform onto which a wheel chair can be rolled and at least one wheel block for preventing movement of the wheels of the wheelchair.

14. The bicycle of claim 8 comprising a screen and a microprocessor adapted to display and log data regarding the therapy of the patient and transmit that data via a telecommunications link to a therapist.

15. The bicycle of claim 8 comprising a screen, a microprocessor, and a digital camera adapted to transmit images of the person to a third party and to receive images of the third party and display the images on the screen.

16. The bicycle of claim 8 comprising a screen and a microprocessor adapted to generate and display a virtual image of the user using the bicycle in a virtual location.

17. The bicycle of claim 8 comprising a microphone and a microprocessor adapted to receive voice commands from the person and control the bicycle in response thereto.

18. The bicycle of claim 8 comprising a screen and a microprocessor implementing a personal computer operating system to allow the person to perform the functions selected from the list consisting of: browsing the Internet, sending email, drafting letters, playing games and tracking personal finances while the person's limbs are being exercised.
